(19) United States
(12) Patent Application Publication
(10) Pub. No.: US 2018/0339963 A1
(43) Pub. Date: Nov. 29, 2018

(54) ANTICONVULSANT CARBAMATE DERIVATIVES

(71) Applicant: Yissum Research Development Company of the Hebrew University of Jerusalem Ltd., Jerusalem (IL)

(72) Inventors: Meir BIALER, Jerusalem (IL); David BIBI, Petach Tikva (IL); Hafiz MAWASI, Baqa Al-Garbiya (IL)

(73) Assignee: Yissum Research Development Company of the Hebrew University of Jerusalem Ltd., Jerusalem (IL)

(21) Appl. No.: 15/986,295

(22) Filed: May 22, 2018

Related U.S. Application Data
(60) Provisional application No. 62/509,886, filed on May 23, 2017.

(51) Int. Cl. C07C 311/47; A61P 25/14

(52) U.S. Cl. CPC C07C 311/47; A61P 25/14

(57) ABSTRACT

The invention contemplates novel materials as anticonvulsant agents.

### TECHNOLOGICAL FIELD

The invention generally relates to novel carbamate compounds and uses thereof as anticonvulsant drugs.

### BACKGROUND ART

Epilepsy is one of the most common neurological diseases, with between 34-76 per 100,000 people developing epilepsy annually. Epilepsy therapy for the past 100 years is based on the use of antiepileptic drugs (AEDs), which can be divided into two generations; First generation includes AEDs approved before 1990 and include four first-line AEDs: carbamazepine, phenobarbital, phenytoin and valproic acid (VPA) and the second generation includes the AEDs that have been introduced after 1990. Nevertheless, despite the availably of more than 20 old and new AEDs, approximately 30% of patients with epilepsy are not seizure-free using the existing medications. Furthermore, the clinical use of many existing AEDs is restricted by their side effects, including the teratogenicity associated with the therapeutic use of VPA that restricts its use in women of child-bearing age. Consequently, there is an unmet clinical need to discover and develop novel chemical entities that may offer more efficacious and safer therapeutic options over existing AEDs.

In recent years, extensive structure-anticonvulsant activity relationship (SAR) studies of numerous branched and non-branched aliphatic carboxylic acids have been performed [1-6]. Octanoic acid, like other non-branched short fatty acids, were found to be inactive in anticonvulsant tests with the exception of decanoic acid which was recently found to be active through direct AMPA receptor inhibition [7,8].

VPA (isoocatnoic acid) is an achiral branched monocarboxylic acid. The presence of eight carbons in its chemical structure gives VPA an optimal balance between anticonvulsant activity and sedative/hypnotic adverse effects, since efficacy and the sedative side effect increase in VPA homologues with more than 8 carbons [9,10].

Acetazolamide is an old AED, structurally containing a sulfonamide group in its structure [11]. In the late 1970s Ganz and colleagues evaluated the anticonvulsant activity of nine derivatives of 4-amino-benzenesulfonamide (sulfanilamide) in electrically-(MES) and chemically-induced (scMet) seizure tests and found that they displayed an excellent anticonvulsant activity profile [12]. Syntheses of various aromatic sulfonamide derivatives demonstrated the impact of different sulfonamide moieties on their anticonvulsant activity [12,13]. Tasso et al., have reported that 4-(valproylamido)-benzenesulfonamide is a potent anticonvulsant in the mouse-MES test [14].

Hen et al., synthesized and evaluated the anticonvulsant profile and teratogenicity of new amide derivatives of branched aliphatic carboxylic acids with 4-aminobenzensulfonamide. Three (sulfamoylphenyl) butyramide derivatives were the most potent compounds possessing in rats MES-ED₅₀ values of 7.6 mg/kg, 9.9 mg/kg and 9.4 mg/kg [15].

Many carbamate compounds have demonstrated potential therapeutic uses. The discovery in the 1960s that the old dicarbamate anxiolytic drug meprobamate also possessed anticonvulsant activity prompted the design and anticonvulsant evaluation of several anticonvulsant carbamates [16]. SAR studies of carbamates showed that derivatives with two alkyl groups attached at C-2 possess stronger muscle paralyzing activity, whereas the presence of a phenyl group at the 2 position enhances anticonvulsant efficacy [17].

Cenobamate (YKP3089) is a new carbamate currently undergoing Phase II/III clinical trials in epilepsy. At all doses tested (100-400 mg) this compound was highly effective compared to placebo in reducing the frequency of partial-onset seizures on traditional efficacy endpoints [18]. Cenobamate at 200 and 400 mg was highly effective compared to placebo in achieving seizure freedom as an adductive therapy in patients with refractory partial onset seizures [19]. An open label safety study with a slower initial titration rate is currently ongoing. Hen et al., designed and comparatively evaluated the anticonvulsant activity of a series of 19 branched alkyl and aryl carbamates, many of which were VPA derivatives [20]. Subsequently, Shekh-Ahmad et al., evaluated the enantioselective pharmacodynamics and pharmacokinetics of the most active chiral carbamates [21].

### REFERENCES

[1] Bialer M, Yagen B (2007) Valproic Acid: second generation. Neurotherapeutics 4: 130-137.
[2] Liu M J, Pollack G M (1994) Pharmacokinetics and pharmacodynamics of valproate analogues in rats. IV. Anticonvulsant action and neurotoxicity of octanoic acid, cyclohexanecarboxylic acid, and 1-methyl-1-cyclohexanecarboxylic acid. Epilepsia 35:234-243.
[3] Loscher W, Nau H (1985) Pharmacological evaluation of various metabolites and analogues of valproic acid. Anticonvulsant and toxic potencies in mice. Neuropharmacology 24:427-435.
[4] Bialer M (2012) Chemical properties of antiepileptic drugs (AEDs). Adv. Drug Deliv. Rev. 64:887-895.
[5] Rogawski M A (2016) A fatty acid in MCT ketogenic diet for epilepsy treatment blocks AMPA receptors. Brain 139:306-309.
[6] Keane P E, Simiand J, Mendes E, Santucci V, Morre M (1983) The effects of analogues of valproic acid on seizures induced by pentylenetetrazol and GABA content in brain of mice. Neuropharmacology 22:875-879.
[7] Resor A S R, Resor L D, Woodbury D M, Kemp J W (1995) Acetazolamide. In: Levy R H, Mattson R H, Meldrum B S (eds) Antiepileptic Drugs, 4th edn, Raven Press, New York, pp 969-985.
[8] Ganz A J, Waser P G, Pfirrmann R W (1978) Development of new antiepileptics. V. Pharmacological activity of some derivatives of sulfanilamide (author's transl). Arzneimittelforschung 28:1331-1334.
[9] Masereel B, Rolin S, Abbate F, Scozzafava A, Supuran C T (2002) Carbonic anhydrase inhibitors: anticonvulsant sulfonamides incorporating valproyl and other lipophilic moieties. J. Med. Chem. 45:312-320.
[10] Tasso S M, Moon S, Bruno-Blanch L E, Estiu G L (2004) Characterization of the anticonvulsant profile of valpromide derivatives. Bioorg. Med. Chem. 12:3857-3869.
[11] Hen N, Bialer M, Wlodarczyk B, Finnell R H, Yagen B (2010) Syntheses and evaluation of anticonvulsant profile and teratogenicity of novel amide derivatives of branched aliphatic carboxylic acids with 4-aminobenzensulfonamide. J Med Chem. 53:4177-4186.
[12] Murray W J, Kier L B (1977) Noncyclic anticonvulsants. In: Vida J A (ed) Medicinal Chemistry, Academic Press, New York, pp 578-619.
[13] Ludwing B J, Powell L S, Berger F M (1969) Carbamate derivatives related to meprobamate. J Med Chem 12:462-472.
[14] Bialer M, Johannessen S I, Levy R H, Perucca E, Tomson T, White H S (2015) Progress report on new antiepileptic drugs: a summary of the Twelfth Eilat Conference (EILAT XII). Epilepsy Res 111:85-141.
[15] Murray W J, Kier L B (1977) Noncyclic anticonvulsants. In: Vida J A (ed) Medicinal Chemistry, Academic Press, New York, pp 578-619.
[16] Hen N, Bialer M, Yagen B (2012) Syntheses and evaluation of anticonvulsant activity of novel branched alkyl carbamates. J Med Chem 55:2835-2845.
[17] Shekh-Ahmad T, Mawasi H, McDonough J H, Finnell R H, Wlodarczyk B J, Bialer M (2014) Enantioselective pharmacodynamic and pharmacokinetic analysis of two active chiral CNS-active carbamate derivatives of valproic acid. Epilepsia 55:1944-1952.
[18] White H S, Woodhead J H, Wilcox K S, Stables J P, Kupferberg H J, Wolf H H (2002) Discovery and Preclinical Development. In: Levy R H, Mattson R H, Meldrum B S, Perucca E (eds), Antiepileptic Drugs, 5th edn, Lippincott Williams & Wilkins, New York, pp 36-48.
[19] Smith M, Wilcox K S, White H S (2007) Discovery of antiepileptic drugs. Neurotherapeutics 4:12-17.
[20] Wlodarczyk B C, Craig J C, Bennett G D, Calvin J A, Finnell R H (1996) Valproic Acid-Induced Changes in Gene Expression During Neurulation in a Mouse Model. Teratology 45:284-297.
[21] Khalifah R G (1971) The carbon dioxide hydration activity of carbonic anhydrase. I. Stop-flow kinetic studies on the native human isoenzymes B and C. J. Biol. Chem. 246:2561-2573.
[22] Casini A, Antel J, Abbate F, Scozzafava A, David S, Waldeck H, Schafer S, Supuran C T (2003) Carbonic anhydrase inhibitors: SAR and X-ray crystallographic study for the interaction of sugar sulfamates/sulfamides with isozymes I, II and IV. Bioorg. Med. Chem. Lett. 13:841-845.
[23] De Simone G, Di Fiore A, Menchise V, Pedone C, Antel, J, Casini A, Scozzafava A, Wurl M, Supuran C T (2005) Carbonic anhydrase inhibitors. Zonisamide is an effective inhibitor of the cytosolic isozyme II and mitochondrial isozyme V: solution and X-ray crystallographic studies. Bioorg. Med. Chem. Lett. 15:2315-2320;
[24] Temperini C, Innocenti A, Scozzafava A, Parkkila S, Supuran C T (2010) The coumarin-binding site in carbonic anhydrase accommodates structurally diverse inhibitors: the antiepileptic lacosamide as an example. J. Med. Chem. 53:850-854.
[25] Supuran C T (2008) Carbonic anhydrases: Novel Therapeutic Applications for Inhibitors and Activators. Nature Rev. Drug Discov. 7:168-181.
[26] Jezequel S G (1992), General Nervous System Penetration of Drugs: Importance of Phisicochemical Properties, Taylor & Francis London, pp 141-178.
[27] Pitea M, M\check{a}rie A, Cristea R, T\check{a}maş M, Arieşan V (1973), Investigations on some sulfamidourethanes. Archiv der Pharmazie 306(9):702-6.

### GENERAL DESCRIPTION

There is a critical need to discover novel chemical entities for the development of safe and potent AEDs. The inventors of the present invention assessed the effect of changing the length and the branching of the aliphatic side chains in the alcohols used for the derivatization (esterification) with 4-aminobenzenesulfonamide-carbamic acid and designed and comparatively analyzed the anticonvulsant activity, neurotoxicity, teratogenicity and carbonic anhydrase (CA) inhibition of new carbamate derivatives of 4-aminobenzenesulfonamide (FIG. 1), possessing 6-9 carbons in the aliphatic side chain of the carbamate moieties. The teratogenicity of the most potent anticonvulsant compounds was evaluated in mice and their carbonic anhydrase (CA) inhibition of four human-CA isoforms was tested in vitro compared to that of acetazolamide. The structure-activity-relationship (SAR) of the above mentioned carbamates was determined for developing new potent and safe AEDs.

Thus, in one of its aspects the present invention provides a compound having a 4-sulfamoylphenyl carbamate moiety and an alkyl moiety comprising between 5 and 10 carbon atoms.

The 4-sulfamoylphenyl carbamate moiety having the structure:

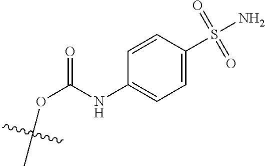 [SMILES: *OC(=O)Nc1ccc(S(N)(=O)=O)cc1]

wherein the wavy line designates the point of connection (or bond connecting) to the alkyl moiety; and wherein the amine group of the 4-sulfamoylphenyl may be hydrogenated or further alkylated to provide an alkylated or non-alkylated ammonium group (NH₃⁺, NH₂R, NHRR′, NRR′R″, wherein each R group, independently, may be an alkyl group having between 1 and 4 carbon atoms).

The alkyl moiety comprising between 5 and 10 carbon atoms may be any straight or branched alkyl group comprising 5, 6, 7, 8, 9, or 10 carbon atoms. In some embodiments, the alkyl moiety is selected from substituted or unsubstituted pentyl moiety, substituted or unsubstituted hexyl moiety, substituted or unsubstituted heptyl moiety, substituted or unsubstituted octyl moiety, substituted or unsubstituted nonyl moiety or substituted or unsubstituted decyl moiety, provided that the number of carbon atoms in the moiety does not exceed 10 atoms. In some embodiments, substitution is by an alkyl group having between 1 and 5 carbon atoms. In some embodiments, the substitution is by an aryl group, e.g., a phenyl group.

In some embodiments, the alkyl moiety is selected from 3-methyl-2-propylpentyl, 2-ethylhexyl, 2-propylpentyl, 3,3-dimethylbutyl, phenethyl, 2-ethylbutyl, 2,4,4-trimethylpentyl, 2,4-dimethylpentan-3-yl, 3-methylpentan-2-yl and 3-methylpentyl.

In some embodiments, the compound of the invention is of the formula (I):

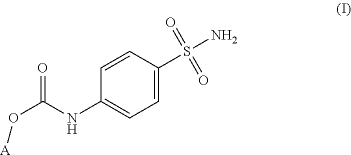

wherein

A is an alkyl moiety, as defined, bonded to the oxygen atom of the 4-sulfamoylphenyl carbamate directly, as shown, or via a linker moiety L, as shown in formula (II):

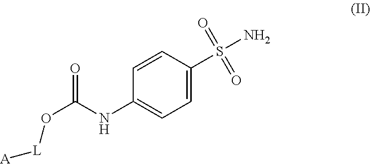

wherein

A is an alkyl moiety, as defined, and L is a linker moiety selected from an atom or a group of atoms, e.g., alkylene having between 1 and 5 carbon atoms, —O-alkylene, alkylene-O-alkylene and others.

In some embodiments, L is absent and A is connected directly to the O atom of the 4-sulfamoylphenyl carbamate.

In some embodiments, the compound of the invention is selected from compounds herein designated (1) through (10), an enantiomer, prodrug, a hydrate, a solvate or a pharmaceutically acceptable salt thereof:
3-methyl-2-propylpentyl (4-sulfamoylphenyl)carbamate (1);
2-ethylhexyl (4-sulfamoylphenyl)carbamate (2);
2-propylpentyl (4-sulfamoylphenyl)carbamate (3);
3,3-dimethylbutyl (4-sulfamoylphenyl)carbamate (4);
phenethyl (4-sulfamoylphenyl)carbamate (5);
2-ethylbutyl (4-sulfamoylphenyl)carbamate (6); (7);
2,4,4-trimethylpentyl (4-sulfamoylphenyl)carbamate (7);
2,4-dimethylpentan-3-yl (4-sulfamoylphenyl)carbamate (8);
3-methylpentan-2-yl (4-sulfamoylphenyl)carbamate (9);
3-methylpentyl(4-sulfamoylphenyl)carbamate (10).

In some embodiments, the compound of the invention is the compound herein designated (1), (2), (3), (4), (5), (6), (7), (8), (9) or (10).

In some embodiments, the compound is a compound herein designated (1) or (9) or (10).

As a person skilled in the art would realize, compounds of the invention contain at least one chiral center. Some compounds of the invention contain two or more chiral centers. Thus, the present invention also provides specific stereoisomers of any one of the aforementioned compounds.

The invention thus provides the compounds in any one enantiomerically pure form, or as stereoisomeric or diastereomeric mixtures. It is to be understood that the chiral centers of the compounds provided herein may undergo epimerization in vivo. As such, one of skill in the art will recognize that, for example, administration of a compound in its (R) form is equivalent, for compounds that undergo epimerization in vivo, to administration of the compound in its (S) form. Where multi-chiral centers are concerned, the same rules may apply.

In another one of its aspects, the present invention provides use of a compound in the preparation of a composition, the compound being selected from
3-methyl-2-propylpentyl (4-sulfamoylphenyl)carbamate (1);
2-ethylhexyl (4-sulfamoylphenyl)carbamate (2);
2-propylpentyl (4-sulfamoylphenyl)carbamate (3);
3,3-dimethylbutyl (4-sulfamoylphenyl)carbamate (4);
phenethyl (4-sulfamoylphenyl)carbamate (5);
2-ethylbutyl (4-sulfamoylphenyl)carbamate (6);
2,4,4-trimethylpentyl (4-sulfamoylphenyl)carbamate (7);
2,4-dimethylpentan-3-yl (4-sulfamoylphenyl)carbamate (8);
3-methylpentan-2-yl (4-sulfamoylphenyl)carbamate (9);
3-methylpentyl(4-sulfamoylphenyl)carbamate (10);
sec-butyl(4-sulfamoylphenyl)carbamate (11).

In some embodiments, the composition is a pharmaceutical composition.

In another aspect, there is provided use of a compound in a method of treatment, the compound being selected from
3-methyl-2-propylpentyl (4-sulfamoylphenyl)carbamate (1);
2-ethylhexyl (4-sulfamoylphenyl)carbamate (2);
2-propylpentyl (4-sulfamoylphenyl)carbamate (3);
3,3-dimethylbutyl (4-sulfamoylphenyl)carbamate (4);
phenethyl (4-sulfamoylphenyl)carbamate (5);
2-ethylbutyl (4-sulfamoylphenyl)carbamate (6);
2,4,4-trimethylpentyl (4-sulfamoylphenyl)carbamate (7);
2,4-dimethylpentan-3-yl (4-sulfamoylphenyl)carbamate (8);
3-methylpentan-2-yl (4-sulfamoylphenyl)carbamate (9);
3-methylpentyl(4-sulfamoylphenyl)carbamate (10); and
sec-butyl(4-sulfamoylphenyl)carbamate (11).

The present invention further provides a composition comprising at least one compound, alone or in combination with one or more additional compound, the at least one compound being selected from
3-methyl-2-propylpentyl (4-sulfamoylphenyl)carbamate (1);
2-ethylhexyl (4-sulfamoylphenyl)carbamate (2);
2-propylpentyl (4-sulfamoylphenyl)carbamate (3);
3,3-dimethylbutyl (4-sulfamoylphenyl)carbamate (4);
phenethyl (4-sulfamoylphenyl)carbamate (5);
2-ethylbutyl (4-sulfamoylphenyl)carbamate (6);
2,4,4-trimethylpentyl (4-sulfamoylphenyl)carbamate (7);
2,4-dimethylpentan-3-yl (4-sulfamoylphenyl)carbamate (8);
3-methylpentan-2-yl (4-sulfamoylphenyl)carbamate (9);
3-methylpentyl(4-sulfamoylphenyl)carbamate (10); and/or
sec-butyl(4-sulfamoylphenyl)carbamate (11).

In some embodiments, the one or more additional compound may be similarly selected from compounds used in accordance with the invention or any other drug or medicament.

In some embodiments, the composition is a pharmaceutical composition and the compound comprised therein as an active ingredient is a compound herein designated (1), (2), (3), (4), (5), (6), (7), (8), (9), (10) or (11). In some embodiments, the compound is a compound herein designated (1) or (9) or (10) or (11).

In some embodiments, the compound used in accordance with the invention is not compound herein designated (1) or (2) or (3) or (4) or (5) or (6) or (7) or (8) or (9) or (10) or (11). In some embodiments, the compound used in accordance with the invention is not compound herein designated (11).

The pharmaceutical composition according to the present invention may comprise, in addition to compound (1), (2), (3), (4), (5), (6), (7), (8), (9), (10) or (11) one or more pharmaceutically acceptable carrier, vehicle, adjuvant, excipient, or diluent, as known in the art. The pharmaceutically acceptable carrier(s) is selected to be chemically inert to the active compound(s) contained in the composition, and has no detrimental side effects or toxicity under the conditions of use.

The choice of carrier will be determined in part by the particular compound of the invention as well as by the particular method used to administer the composition. Accordingly, there is a wide variety of suitable formulations of the pharmaceutical composition of the present invention; these formulations include formulations for oral, aerosol, parenteral, subcutaneous, intravenous, intramuscular, interperitoneal, rectal, and vaginal administration.

The present invention further concerns a method of treatment of a neurological disease or disorder, the method comprising administering to a subject in need of such treatment an effective amount of a compound according to the invention or any pharmaceutical composition comprising same.

As used herein the “neurological diseases or disorder” is selected, in a non-limiting fashion, from epilepsy, convulsions, seizure disorder, complex partial seizures, status epilepticus, a chemically-induced convulsion and/or seizure disorder, a febrile convulsion condition, pain (particularly neuropathic pain, differentiation pain, migraine and headaches) and psychiatric disorders (particularly schizophrenia, bipolar disorder and anxiety).

Some further non-limiting examples of neurological diseases or disorders, in accordance with the present invention, are selected from epilepsy, status epilepticus, chemically-induced convulsion and/or seizure disorder (e.g., induced by chemical warfare), neurophatic pain and bipolar disorders.

The “effective amount” of a compound according to the present invention, or a composition comprising thereof according to the invention, used for purposes herein, is determined by such considerations as may be known in the art. The amount must be effective to achieve a desired therapeutic effect as described above, e.g., treatment and/or prevention of epilepsy depending, inter alia, on the type and severity of the disease to and the existing treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

The term “treatment” may refer to a decrease in the duration and/or severity of the acute phase of the disease or disorder (decrease in the duration and/or in the severity of the epileptic attack, chemically induced convulsion attack, migraine attack acute phase of bipolar, e.g., manic phase, etc.). The term also encompasses prevention, either of said acute phases altogether (preventing epileptic attacks, migraine, and prevention of chemically induced seizure or bipolar phases) or decreasing the incidence of the acute phase.

In some embodiments, treatment refers to an anticonvulsant treatment.

As used herein, the term “anticonvulsant” generally refers to an activity that stops and/or ameliorates an ongoing (e.g. epileptic) seizure or decreases the frequency or severity of anticipated future seizures.

In some embodiments, the herein described seizure is status epilepticus (SE) defined as a continuous seizure lasting at least 5 minutes (and in some cases more than 2 minutes) and typically more than 30 minutes or two or more seizures without full recovery of consciousness between any of them. Prolonged SE can lead to cardiac dysrhythmia, metabolic derangements, autonomic dysfunction, neurogenic pulmonary edema, hyperthermia, rhabdomyolysis, and pulmonary aspiration. Permanent neurologic damage can occur with prolonged SE.

The present invention further relates to a method for the (e.g. stereoselective) preparation of a compound of the invention, the method comprising contacting an alkyl alcohol under suitable conditions with 4-aminobenzenesulfonamide to obtain a compound of the invention. The alkyl alcohol being an alkanol comprising an alkyl moiety selected for the specific compound, and defined as herein.

In some embodiments, the alkyl alcohol is first reacted with phosgene to afford the acyl chloride which is then reacted with the 4-aminobenzenesulfonamide. This method is depicted in Scheme 1:

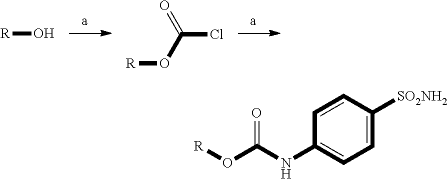

wherein

R is the alkyl moiety as defined herein, and the reaction conditions are, in some embodiments: (a) treating the alkyl alcohol with triphosgene; (b) treating the resulting acyl chloride with 4-aminobenzenesulfonamide.

In an alternative method, the compounds of the invention are obtained, as shown in Scheme 2:

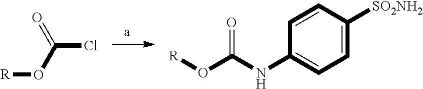

wherein

R is the alkyl moiety as defined herein, and the acyl chloride, formed according to any procedure known in the art, is reacted with 4-aminobenzenesulfonamide under suitable conditions.

In some embodiments, the reactions are carried out at room temperature.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts the structures of exemplary compounds of the invention.

### DETAILED DESCRIPTION OF EMBODIMENTS

A novel series of carbamate derivatives containing 4-aminobenzenesulfonamide and branched VPA or phenethyl moieties were synthesized in good yields and screened for their anticonvulsant activities in MES and scMet and 6 Hz test. In mice compounds 1, 9, 10 and 11 had ED₅₀ values of 236 mg/kg, 31 mg/kg, 14 mg/kg and 75 mg/kg (MES) and 74 mg/kg, 53 mg/kg, 88 mg/kg (6 Hz), respectively. Compounds 1, 9, 10 and 11 had rat (po)-MES-ED₅₀ values of 36 mg/kg, 28 mg/kg, 23 mg/kg and 25 mg/kg, respectively. These potent carbamates induced neural tube defects only at doses markedly exceeding their anticonvuslnat-ED₅₀ values. None of these compounds were potent inhibitors of CA IV, but inhibited isoforms CAs I, II and VII. All three of these compounds (and particularly compound 10) exhibited better protective index than VPA.

Materials and Methods

Chemistry.

Unless otherwise stated, all reagents were purchased from commercial suppliers and used without further purification. Solvents used in the reactions were distilled from appropriate drying agents prior to use.

General Procedure for the Synthesis of Compounds.

The general syntheses for the 4-aminobenzenesulfonamides derivatives in Scheme 1. A solution of the alcohol (1 equiv) in dichloromethane (DCM, 5 mL) was added dropwise to a stirred solution of triphosgene (0.5 equiv) and pyridine (1 equiv) in DCM (10 mL). The reaction mixture was allowed to stir for 2 hr and then was evaporated. The residue was dissolved in THF (10 mL). A solution of sulfanilamide (500 mg) in THF (10 mL) was added and stirred overnight. The reaction was quenched with water and extracted with EtOAc (15 mL). The organic layer was washed with water and brine, dried over sodium sulfate, filtered, and concentrated in vacuo. The desired product was obtained following flash chromatography (EtOAc/hexane). The compounds designed in this study were synthesized by non-stereospecific methods and thus evaluated as racemates.

### 3-methyl-2-propylpentyl (4-sulfamoylphenyl)carbamate (1)

White powder; mp 151-152° C. ¹H NMR (300 MHz, DMSO-d₆) δ 9.94 (s, 1H), 7.76-7.65 (m, 2H), 7.65-7.54 (m, 2H), 7.19 (s, 2H), 4.15-3.92 (m, 2H), 3.36-3.23 (m, 1H), 1.56-1.04 (m, 7H), 0.92-0.77 (m, 9H). Calcd for C16H26N2O4S: C, 56.12; H, 7.65; N, 8.18; S, 9.36. Found: C, 56.12; H, 7.76; N, 8.0; S, 9.08.

### 2-ethylhexyl (4-sulfamoylphenyl)carbamate (2)

White powder; mp 192-195° C. ¹H NMR (300 MHz, DMSO-d₆) δ, 8.10 (s, 1H), 7.39 (d, J=8.6 Hz, 2H), 7.26 (s, 2H), 7.04 (d, J=8.6 Hz, 1H), 4.29 (m, 2H), 1.62-1.68 (m, 1H), 1.44-1.23 (m, 8H), 0.99-0.80 (m, 6H). Calcd for C15H24N2O4S: C, 58.86; H, 7.37; N, 8.53; S, 9.76. Found: C, 56.62; H, 7.09; N, 8.57; S, 9.58.

### 2-propylpentyl (4-sulfamoylphenyl)carbamate (3)

White powder; mp 176-177° C. ¹H NMR (300 MHz, DMSO-d₆) δ 9.92 (s, 1H), 7.76-7.65 (d, J=8.6 Hz, 2H), 7.65-7.54 (d, J=8.7 Hz, 1H), 7.17 (s, 2H), 4.00 (m, 2H), 1.66 (s, 1H), 1.42-1.18 (m, 8H), 0.97-0.81 (m, 6H). Calcd for C15H24N2O4S: C, 54.86; H, 7.37; N, 8.53; S, 9.76. Found: C, 55.05; H, 7.28; N, 8.43; S, 9.54.

### 3,3-dimethylbutyl (4-sulfamoylphenyl)carbamate (4)

White powder; mp 170-173° C. ¹H NMR (300 MHz, DMSO-d₆) δ 9.99 (s, 1H), 7.67-7.74 (d, J=8.7 Hz, 2H), 7.63-7.5 (d, J=8.7 Hz, 2H), 7.21 (s, 2H), 4.11 (m, 2H), 1.57-1.48 (m, 2H) 0.96 (s, 9H). Calcd for C13H20N2O4S: C, 51.98; H, 6.71; N, 9.33; S, 10.68. Found: C, 52.03; H, 6.66; N, 9.2; S, 10.33.

### phenethyl (4-sulfamoylphenyl)carbamate (5)

White powder; mp 212-213° C. ¹H NMR (300 MHz, DMSO-d₆) δ 10.02 (s, 1H), 7.56-7.63 (d, J=8.6 Hz, 2H), 7.68-7.72 (d, J=8.6 Hz, 2H), 7.29 (s, 2H), 7.31-7.19 (m, 5H), 4.41 (t, J=6.8 Hz, 2H), 2.84 2.95 (t, J=6.8 Hz, 2H). Calcd for C15H16N2O4S: C, 56.24; H, 5.03; N, 8.74; S, 10.01. Found: C, 55.9; H, 5.07; N, 8.61; S, 9.64.

### 2-ethylbutyl (4-sulfamoylphenyl)carbamate (6)

White powder; mp 162-165° C. ¹H NMR (300 MHz, DMSO-d₆) δ 9.92 (s, 1H), 7.58-7.62 (d, J=8.9 Hz, 2H), 7.68-7.72 (d, J=8.9 Hz, 2H), 7.21 (s, 2H), 4.02 (m, 2H), 2.07 (s, 1H), 1.36 (m, 1H), 1.05-0.87 (m, 6H). Calcd for C13H20N2O4S: C, 51.98; H, 6.71; N, 9.33; S, 10.68. Found: C, 51.59; H, 6.65; N, 9.18; S, 10.54.

### 2,4,4-trimethylpentyl (4-sulfamoylphenyl)carbamate (7)

White powder; mp 199-201° C. ¹H NMR (300 MHz, DMSO-d₆) δ 9.96 (s, 1H), 7.76-7.65 (d, J=8.7 Hz, 1H), 7.65-7.54 (d, J=8.7 Hz, 1H), 7.18 (s, 2H), 3.97 (m, 1H), 3.79 (m, 1H), 1.83 (m, 1H), 1.92-1.76 (m, 1H), 1.29 (m, 1H), 1.00 (m, 4H), 0.89 (s, 8H). Calcd for C15H24N2O4S: C, 54.86; H, 7.37; N, 8.53; S, 8.76. Found: C, 54.48; H, 7.29; N, 8.31; S, 8.39.

### 2,4-dimethylpentan-3-yl (4-sulfamoylphenyl)carbamate (8)

White powder; mp 193-194° C. ¹H NMR (300 MHz, DMSO-d₆) δ 9.94 (s, 1H), 7.70 (d, J=8.6 Hz, 2H), 7.60 (d, J=8.7 Hz, 2H), 7.20 (s, 2H), 4.43 (t, J=6.2 Hz, 1H), 1.90 (h, J=7.0 Hz, 2H), 0.87 (d, J=6.7 Hz, 12H). Calcd for C14H22N2O4S: C, 53.48; H, 7.05; N, 8.91; S, 10.20. Found: C, 53.42; H, 7.25; N, 8.62; S, 9.93.

### 3-methylpentan-2-yl (4-sulfamoylphenyl)carbamate (9)

White powder; mp 179-180° C. ¹H NMR (300 MHz, DMSO-d₆) δ 9.90 (s, 1H), 7.57-7.61 (d, J=8.7 Hz, 1H), 7.68-7.71 (d, J=8.7 Hz, 1H), 7.18 (s, 2H), 4.82-4.63 (m, 1H), 1.65-1.41 (m, 1H), 1.17 (m, 5H), 0.88 (m, 6H). Calcd for C13H20N2O4S: C, 51.98; H, 6.71; N, 9.33; S, 10.68. Found: C, 52.03; H, 6.61; N, 9.32; S, 11.2.

### 3-methylpentyl(4-sulfamoylphenyl)carbamate (10)

White powder; mp 168-169° C. ¹H NMR (300 MHz, DMSO-d₆) δ 10.00 (s, 1H), 7.70 (d, J=8.8 Hz, 2H), 7.59 (d, J=8.7 Hz, 2H), 7.20 (s, 1H), 4.18-4.07 (m, 2H), 1.73-1.56 (m, 1H), 1.11-1.21 (m, 4H), 0.85 (m, 6H).

Calcd for C13H20N2O4S: C, 51.98; H, 6.71; N, 9.33; S, 10.68. Found: C, 52.12; H, 6.54; N, 9.35; S, 10.77.

### sec-butyl(4-sulfamoylphenyl)carbamate (11)

White powder; mp 190-191° C. ¹H NMR (300 MHz, DMSO-d₆) δ 9.97 (s, 1H), 7.69 (d, J=8.8 Hz, 2H), 7.60 (d, J=8.7 Hz, 2H), 7.20 (s, 2H), 4.77-4.68 (m, 1H), 1.63-1.53 (m, 2H), 1.23-1.21 (d, 3H), 0.92-0.87 (t, 3H). Calcd for C11H16N2O4S: C, 48.52; H, 5.92; N, 10.29; S, 11.77. Found: C, 48.47; H, 6.02; N, 10.13; S, 11.56.

Materials and Methods.

Product formation follow-up was performed by means of ¹H NMR and TLC. TLC analyses were performed on pre-coated silica gel on aluminum sheets (Kieselgel 60 F254, Merck). ¹H NMR spectra were recorded on a Varian Mercury series NMR 300 spectrometer. Chemical shifts (6 scale) are reported in parts per million (ppm) relative to the indicated reference. Coupling constants are given in Hz. Chemical structure and purity of the compounds newly synthesized was assessed by TLC, ¹H NMR and HPLC. Melting point was determined on a 1002-230 VAC Mel-Temp capillary melting point apparatus.

Biological Testing/Anticonvulsant Activity.

The evaluation of anticonvulsant activity in the maximal electroshock seizure test (MES), subcutaneous metrazol seizure test (scMet) and 6 Hz psychomotor test (6 Hz), corneal kindled mouse (CKM) and the determination of minimal neurotoxicity using the rotarod test and positional sense test and others were performed at the National Institute of Health (NIH)-National Institute of Neurological Disorders and Stroke (NINDS) as a part of the Epilepsy Therapy Screening Program (ETSP) according protocols described in [22] and [23].

Preparation of the Compounds for Testing.

The tested compounds were suspended in 0.5% methylcellulose and administered intraperitoneally (i.p.) to adult male CF no. 1 albino mice (18-25 g) in a volume of 0.01 mL/g body weight and (b) orally to adult male Sprague-Dawley albino rats (100-150 g) in a volume of 0.04 mL per 10 g of body weight. The pentylenetetrazol solution at convulsing dose was prepared by sufficient dissolution of pentylenetetrazol in 0.9% saline to make 0.85% solution for administration to mice and 2.82% solution for administration to rats [22, 23].

Determination of the Median Effective Dose (ED₅₀) and the Median Neurotoxic Dose ((TD₅₀).

Dose (ED₅₀ & TD₅₀).

For the determination of the ED₅₀ by the respective anticonvulsant procedure, doses of the tested compounds were varied until a minimum of 3-4 points was established between the dose level with 0% protection and 100% protection. These data were subjected to the FORTRAN probit analysis program, and the ED₅₀ and 95% confidence intervals were calculated. The TD₅₀ was determined by varying the dose of the tested compounds until four points were established between the dose level that induced no signs of minimal motor impairment in any of the animals and the dose at which all of the animals were considered impaired. The TD₅₀ and the 95% confidence intervals were calculated by FORTRAN probit analysis. The PI values were calculated by dividing the TD₅₀ by the ED₅₀ [22, 23].

Evaluation of Teratogenicity.

The teratogenic properties of the newly designed compounds (1-10) were evaluated in the highly inbred SWV mice strain known for its high susceptibility to VPA-induced neural tube defects (NTDs) [24, 25]. Two month old nulligravid females, were mated overnight with males and examined for the presence of vaginal plugs the following morning, and the onset of gestation was considered to be 10 p.m. of the previous night, the midpoint of the dark cycle. At day 8.5 of gestation, each dam received a single i.p. injection of the test compounds in an equimolar dose of 0.9 or 1.8 mmol/kg and the control dams were injected with vehicle (25% water solution of Cremophore EL, Fluka Biochemica Germany). The volume of injection was 10 μL/g of body weight. The 1.8 mmol/kg dose was selected as this was the highest dose that did not produce overt maternal toxicity except for deep sedation and lack of ambulation lasting for approximately 2 hours post-injection. At gestation day 8.5, the dams were euthanized by carbon dioxide asphyxiation followed by cervical dislocation. After the opening the uterus, the location of all viable fetuses and resorption sites were recorded, and the fetuses were examined for the presence of exencephaly or other gross congenital abnormalities. The teratogenicity data (implantations, resorptions and NTDs) were evaluated for significant differences between the control and treated groups by analyzing the contingency table with Fisher's exact test. Statistical analysis was conducted using GraphPad InStat (version 3.06; GraphPad Software, San Diego, Calif., USA), and the results of all tests were considered to be statistically significant when the p-value was less than 0.05

CA Inhibition.

An Applied Photophysics stopped-flow instrument has been used for assaying the CA catalysed CO₂ hydration activity [26]. Phenol red (at a concentration of 0.2 mM) has been used as indicator, working at the absorbance maximum of 557 nm, with 20 mM Hepes (pH 7.5) as buffer, and 20 mM Na₂SO₄ (for maintaining constant the ionic strength), following the initial rates of the CA-catalyzed CO₂ hydration reaction for a period of 10-100 s. The CO₂ concentrations ranged from 1.7 to 17 mM for the determination of the kinetic parameters and inhibition constants. For each inhibitor at least six traces of the initial 5-10% of the reaction have been used for determining the initial velocity. The uncatalyzed rates were determined in the same manner and subtracted from the total observed rates. Stock solutions of inhibitor (0.1 mM) were prepared in distilled-deionized water and dilutions up to 0.01 nM were done thereafter with the assay buffer. Inhibitor and enzyme solutions were preincubated together for 15 min at room temperature prior to assay, in order to allow for the formation of the E-I complex. The inhibition constants were obtained by non-linear least-squares methods using PRISM 3 and the Cheng-Prusoff equation, as reported earlier and represent the mean from at least three different determinations. All CA isofoms were recombinant ones obtained in-house.

Calculation of C log P.

C log P was calculated by means of ChemDraw-Ultra Software 8.

### Results

Chemistry

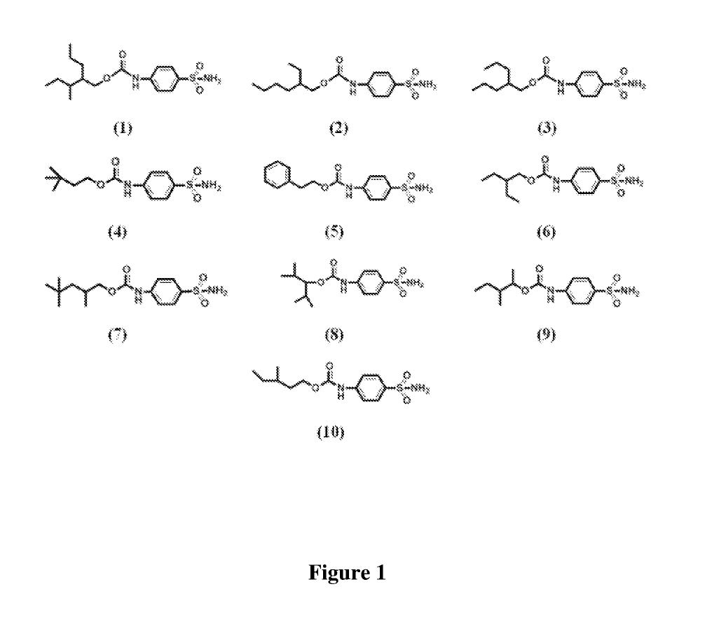

The general synthesis of the carbamates designed in the current study is presented in Scheme 1 and Scheme 2. Compounds (1)-(10) shown in FIG. 1, as well as compounds of Formula (I) were obtained with isolated yields ranging between 62-85%.

Pharmacology

The anticonvulsant activity profile of compounds 1-10 was determined using the MES test, which measures seizure spread, scMet test, which measures seizure threshold and the 6 Hz (32 mA) psychomotor test. These tests are mechanism-independent animal seizure models that enable identification of compounds preventing seizure spread [27]. Compounds 1-10 were administered i.p. to mice and orally (or i.p.) to rats at fixed doses of 30, 100 or 300 mg/kg, and the anticonvulsant protection was observed at different times after dosing. The method applied here allowed the determination of the number of animals (in a group consisting of 4 or 8 mice or rats) protected against electrically- or chemically-induced seizures as well as the estimation of the time course of anticonvulsant activity together with time of peak effect (TPE).

Table 1 presents the anticonvulsant activity and neurotoxicity or minimal behavioral impairment of all tested compounds (compounds 1-10) at the mouse-MES and mouse-6 Hz (32 mA) tests (i.p.). At the MES model, these three most active compounds (compounds 1, 9 and 10) showed some protection at 30 mg/kg and full or almost full (compound 1) protection at 100 mg/kg with no signs of neurotoxicity. These compounds showed partial protection with no neurotoxicity at doses of 30 and 100 mg/kg and full or almost full (compound 1) protection 300 mg/kg at the 6 Hz test with no neurotoxicity.

TABLE 1
Anticonvulsant activity and neurotoxicity (minimal
behavioral impairment) of 4-sulfonamidobenzene
carbamates administered i.p. to mice.

| | Dose | MES^{a} | | 6 Hz^{a, b} | | Tox^{c} | |
|---|---|---|---|---|---|---|---|
| Cmpd | (mg/kg) | 0.5 h^{d} | 2.0 h | 0.5 h | 2.0 h | 0.5 h | 2.0 h |
| 1 | 30 | 0/4 | 0/4 | 1/4 | 0/4 | 0/8 | 0/8 |
| | 100 | 0/4 | 3/4 | 1/4 | 1/4 | 0/8 | 0/8 |
| | 300 | 0/4 | 4/4 | 0/4 | 3/4 | 0/8 | 0/8 |
| 2 | 30 | 0/4 | 0/4 | 0/4 | 0/4 | 0/8 | 0/8 |
| | 100 | 1/4 | 0/4 | 3/4 | 0/4 | 0/8 | 0/8 |
| | 300 | 1/4 | 1/4 | 2/4 | 1/4 | 0/8 | 0/8 |
| 3 | 30 | 0/4 | 0/4 | 0/4 | 0/4 | 0/8 | 0/8 |
| | 100 | 0/4 | 0/4 | 0/4 | 0/4 | 0/8 | 0/8 |
| | 300 | 0/4 | 0/4 | 1/4 | 0/4 | 0/8 | 0/8 |
| 4 | 30 | 0/4 | 0/4 | 0/4 | 0/4 | 0/8 | 0/8 |
| | 100 | 0/4 | 0/4 | 0/4 | 0/4 | 0/8 | 0/8 |
| | 300 | 1/4 | 1/4 | 0/4 | 0/4 | 0/8 | 0/8 |
| 5 | 30 | 0/4 | 1/4 | 0/4 | 0/4 | 0/8 | 0/8 |
| | 100 | 0/4 | 1/4 | 0/4 | 0/4 | 0/8 | 0/8 |
| | 300 | 0/4 | 2/4 | 0/4 | 0/4 | 0/8 | 0/8 |
| 6 | 30 | 0/4 | 0/4 | 0/4 | 0/4 | 0/8 | 0/8 |
| | 100 | 0/4 | 0/4 | 1/4 | 0/4 | 0/8 | 0/8 |
| | 300 | 0/4 | 0/4 | 1/4 | 1/4 | 0/8 | 0/8 |
| 7 | 30 | 0/4 | 0/4 | 1/4 | 0/4 | 0/8 | 0/8 |
| | 100 | 0/4 | 1/4 | 1/4 | 2/4 | 0/8 | 0/8 |
| | 300 | 0/4 | 2/4 | 4/4 | 3/4 | 0/8 | 0/8 |
| 8 | 30 | 0/4 | 2/4 | 0/4 | 0/4 | 0/8 | 0/8 |
| | 100 | 0/4 | 4/4 | 0/4 | 0/4 | 0/8 | 0/8 |
| | 300 | 1/4 | 4/4 | 2/4 | 1/4 | 1/8 | 0/8 |
| 9 | 30 | 3/4 | 0/4 | 2/4 | 0/4 | 0/8 | 0/8 |
| | 100 | 3/4 | 4/4 | 2/4 | 0/4 | 0/8 | 0/8 |
| | 300 | 4/4 | 4/4 | 4/4 | 1/4 | 0/8 | 0/8 |
| 10 | 30 | 3/4 | 1/4 | 1/4 | 0/4 | 1/8 | 0/8 |
| | 100 | 4/4 | 4/4 | 3/4 | 2/4 | 0/8 | 0/8 |
| | 300 | 3/4 | 4/4 | 4/4 | 4/4 | 1/8 | 0/8 |

^{a}Data indicate number of mice protected/number of mice tested. The animals were examined at two pretreatment times: 0.5 and 2 h.
^{b}The 6 Hz psychomotor test was conducted with 32 mA. Maximal electroshock (MES) test (number of animals protected/number of animals tested Neurotoxicity was evaluated as motor impairment or sedation (number of animals affected/number of animals tested). Time after drug administration.

Comparative quantitative evaluation at the mouse-MES, scMet and 6 Hz (32 mA) (i.p.) models showed that compounds 1, 9 and 10 demonstrated anticonvulsant activity in the mouse-MES and 6 Hz models with ED₅₀ values of 136, 31 and 14 mg/kg (MES) and 75 mg/kg, 53 mg/kg and 80 mg/g (6 Hz-32 mA), respectively (Table 2). At the scMet test, compound 10 had an ED₅₀ value of 70 mg/kg however, compounds 1 and 9 had a relatively high ED₅₀ values. Compound 10 had also an ED₅₀ value of 59 mg/kg at the corneal kindled mouse model.

Following oral administration to rats compounds 1, 9 10 had MES-ED₅₀ value of 36 mg/kg, 23 mg/kg, and 28 mg/kg, respectively but were inactive at scMet model. Following i.p. administration to rats compound 10 had an MES-ED₅₀ value of 13 mg/kg, showing similar MES-ED₅₀ values after oral and i.p. administration to rats. At the rat-MES test, compounds 9 and 10 had a wide protective index (PI>19) (Table 3).

TABLE 2
Quantitative anticonvulsant data (ED₅₀, TD₅₀ and PI values) in mice dosed i.p.^{a}

| | MES-ED₅₀ | | scMet-ED₅₀ | | 6 Hz-ED₅₀ | | CKM - ED₅₀ | | Neurox-TD₅₀ |
|---|---|---|---|---|---|---|---|---|---|
| | (mg/kg) | | (mg/kg) | | (mg/kg) | | (mg/kg) | | (mg/kg) |
| Cmpd | [95%CI]^{a} | PI^{b} | [95%CI]^{a} | PI^{b} | [95%CI]^{a} | I^{b} | [95%CI]^{a} | PI^{b} | [95%CI] |
| 1 | 136 | >3.6 | >300 | >1.6 | ^{d}74 | >6.8 | NA | — | >500 |
| | [89-212] | | | | [38-125] | | | | |
| 9 | 31 | >19.4 | 369 | >1.6 | 53^{d} | >11.3 | NA | — | >600 |
| | [20-45] | | [250-527] | | [23-94] | | | | |
| 10 | 14 | 50 | 70 | 10 | 80e | 7.9 | 59 | 11.8 | 698 |
| | [10-21] | | [46-99] | | [50-127] | | [36 -87] | | [457-1678] |

^{a}Median Effective (ED₅₀) and Toxic (TD₅₀) values. In squared parentheses are the 95% confidence intervals (95% CI) determined by probit analysis..
^{c} Protective Index (PI = TD₅₀/ED₅₀).
^{d}The 6 Hz test as done at 32 mA.
eAt the 6 Hz (44 mA)-ED₅₀ = 88 mg/kg [62-135 mg/kg].

TABLE 3
Quantitative anticonvulsant data (ED₅₀, TD₅₀ and
PI values) in rats dosed p.o or i.p.

| | MES- | | scMet | | Neurotoxicity- |
|---|---|---|---|---|---|
| | ED₅₀ | | ED₅₀ | | TD₅₀ |
| | (mg/kg) | | (mg/kg) | | (mg/kg) |
| Compound | [95% CI]^{a} | PI^{b} | [95% CI]^{a} | PIb | [95% CI] |
| 1 (p.o.) | 36 | NA | NA | NA | NA |
| | [25-52] | | | | |
| 9 (p.o.) | 23 | >21.4 | >250 | NA | >500 |
| | [18-31] | | | | |
| 10 (p.o.) | 28 | >17.8 | >250 | NA | >500 |
| | [18-35] | | | | |
| 10 (i.p.) | 13 | >19.2 | NA | NA | >250 |
| | [8-22] | | | | |

^{a}Median Effective (ED₅₀) and Toxic (TD₅₀) values. In squared parentheses are the 95% confidence intervals (95% CI) determined by probit analysis.
^{c}Protective Index (PI = TD₅₀/ED₅₀).

The optimal balance between lipophilic and hydrophilic moieties (log P) is a very important consideration in designing AEDs. The log P values of the synthesized carbamates are depicted in Table 4.

TABLE 4
Lipophilicity data (Clog P) of
the Investigated Compounds.

| Compound | Clog P |
|---|---|
| 1 | 4.0 |
| 2 | 3.6 |
| 3 | 3.6 |
| 4 | 2.4 |
| 5 | 2.1 |
| 6 | 2.6 |
| 7 | 3.4 |
| 8 | 3.4 |
| 9 | 2.3 |
| 10 | 2.6 |

^{a}Clog P was calculated by utilizing the ChemDraw Ultra software, version 12

Teratogenicity

The teratogenicity profile of the active compounds (1, 9 and 10) is presented in Table 5.

These compounds showed some indication of teratogenicity when tested at the 1.8 mmol/kg dose but lacked a teratogenic potential at 0.9 mmol/kg. The 0.9 dosage is 4-10 times higher than their mouse-ED₅₀ values (except for compound 1 mouse-MES-ED₅₀ value) and 12-20 times higher than their rat-MES-ED₅₀ values. The active compounds were embryotoxic as evidenced by the high resorption rate but again at doses significantly higher than their anticonvulsant ED₅₀ values.

TABLE 5
Teratogenic effect in the SWV mouse model of the test compounds

| | Dose mg/kg | No. of | No. of | No. of | No. of live | No. of fetuses | | Fetal weight |
|---|---|---|---|---|---|---|---|---|
| Comp | (mmol/kg) | litters | implants | resorptions | fetuses | with NTDs | | mean ± SD |
| Control | 25% CEL | 11 | 142 | 8 (5.6) | 134 (94.4) | 0 | | 0.95 ± 0.06 |
| 1 | 616 (1.8) | 9 | 109 | 59 (54.1)* | 50 (45.9) | 7 | (14.0)* | 0.88 ± 0.06* |
| 1 | 308 (0.9) | 10 | 126 | 32 (25.4)* | 94 (74.6) | 1 | (1.1) | 0.88 ± 0.03* |
| 9 | 541 (1.8) | 9 | 107 | 58 (54.2)* | 49 (45.8) | 9 | (18.4)* | 0.89 ± 0.08 |
| 9 | 270 (0.9) | 10 | 126 | 17 (13.5)* | 109 (86.5) | 3 | (2.8) | 0.90 ± 0.07 |
| 10 | 541 (1.8) | 9 | 108 | 52 (48.2)* | 56 (51.9) | 17 | (30.4)* | 0.87 ± 0.08* |
| 10 | 270 (0.9) | 11 | 145 | 29 (20.0)* | 116 (80.0) | 9 | (7.8)* | 0.88 ± 0.03* |

*significantly different when compared to the control group For statistical purposes either ANOVA with Tukey post-test multiple comparison (Fetus weight) or Contingency Table Analysis with Fisher's exact test (number of resorptions and NTDs) were performed. P value was set at 0.05

Carbonic Anhydrase (CA) Inhibition

Table 6 shows the inhibition constant (Ki) of compounds 1-10 against four human carbonic anyhydrase (hCA) isoforms. The compounds' potency varied from the low nanomolar to micromolar, depending on the substitution pattern of the aliphatic chain of the carbamates.

TABLE 6
Inhibition data of human CA isoforms hCA I, II, IV
and VII of compounds 1-10 and compared to the
standard sulfonamide inhibitor acetazolamide
(AAZ) utilizing a stopped flow CO₂ hydrase assay.

| | K₁ (nM) | | | |
|---|---|---|---|---|
| Compound | hCAI | hCA II | hCA IV | hCA VII |
| 1 | 89.0 | 5.7 | >10000 | 865.1 |
| 2 | 421.2 | 8.7 | >10000 | >10000 |
| 3 | 484.7 | 19.6 | >10000 | >10000 |
| 4 | 59.5 | 4.5 | 1836.3 | 44.4 |
| 5 | 5.9 | 0.6 | 302.9 | 24.5 |
| 6 | 169.5 | 5.2 | 1690.8 | 52.0 |
| 7 | 83.5 | 5.0 | 2756.6 | 712.3 |
| 8 | 21.0 | 3.7 | 452.4 | 35.4 |
| 9 | 72.9 | 5.4 | 182.6 | 35.5 |
| 10 | 77.0 | 7.6 | 750.0 | 350.6 |
| AAZ | 250 | 12 | 74 | 2.5 |

* Mean from 3 different assays, by a stopped flow technique (errors were in the range of ±5-10% of the reported values)

In the present invention, a novel class of 4-aminobenzenesulfonamide-carbamates incorporating phenethyl- or branched alkyls with 6-8 carbon atoms in their side-chain were synthesized and evaluated for anticonvulsant activity, teratogenicity and CA inhibition.

Three of the ten synthesized new carbamates showed anticonvulsant activity at the MES and 6 Hz tests in mice or rats. In mice compounds 1, 9 and 10 had ED₅₀ values of 236 mg/kg, 31 mg/kg and 14 mg/kg (MES) and 74 mg/, 53 mg/kg and 88 mg/kg (6 Hz), respectively. It worth emphasizing that compound 10 had similar ED₅₀ at the 6 Hz (32 and 44 mA) tests. Compound 10 had ED₅₀ values of 13 mg/kg and 59 mg/kg at the rat-MES the mouse corneal kindling tests. Compound 10-PI values was 59 and above >10 mouse and rat MES test, respectively. The aliphatic moiety of compound 1 is an alcoholic congener of sec-butylpropylacetamdie (SPD) a very potent antiepileptic and CNS compound. Compounds 9 and 10 are two constitutional isomers containing, 1,2-dimethybutyl and 3-methylpentyl, respectively in their aliphatic side-chain.

Comparative analysis of the most active 4-aminobenzenesulfonamide-carbamates designed in this study with the most active 4-aminobenzenesulfonamide amide derivatives synthesized and evaluated in a previous study [18], yields the followings: a) The three most active compounds contained branched butyl or pentyl moieties in their aliphatic side-chain and had similar rat-MES-ED₅₀ values, ranging from 7.7-23 mg/kg. b) A dimethylbutyl side-chain leads to an active anticonvulsant compound in both the amide and carbamate groups with rat-MES-ED₅₀ values of 9.4 and 28 mg/kg, respectively. Compound 10-ED₅₀ and wide PI values were similar those of the best 4-aminobenzenesulfonamide amide derivatives described previously [21]. The log P values of the most active amides ranged between 1.0-1.5 while the log P values of the most active carbamates ranged between 2.3-4, indicating that a higher (more lipophylic) log P value does not lead directly to a more potent anticonvulsant activity. The active branched aliphatic carbamoyl-sulfonamides 1, 9 and 10 had similar log P values as the other less-active compounds. These results imply that BBB (Blood-Brain Barrier) penetration due to lipophilicity is an important factor in affecting the test drugs anticonvulsant efficacy but not the only one.

These potent carbamates (1, 9 and 10) induced neural tube defects only at doses markedly exceeding their effective dose. None of these compounds were potent inhibitors of carbonic anhydrase IV, but did inhibit isoforms CAs I, II and VII.

The following SAR can be drawn from the CA inhibition data depicted in Table 6. The isoform CA I was inhibited with potencies ranging between 5.9 and 485 nM by the investigated sulfonamide carbamates, with good inhibition observed for compounds 5 and 8 which incorporate the phenethyl moiety (5, K_{I} of 5.9 nM) and a highly branched aliphatic chain (8, K_{I} of 21 nM).

The carbamates inhibited CA II with K_{I} values comparable or better to the clinically used standard acetazolamide, with the exception of phenethyl carbamate (5) that was endowed with a Ki value 20 times lower than acetazolamide, (K_{I} of 0.6 nM). The three compounds that showed in vivo anticonvulsant activity were all highly effective hCA II inhibitors KI values of 5.4-7.6 nM (better than acetazolamide).

The transmembrane isoform CA IV was not or was poorly inhibited by the designed carbamates. Indeed, only compounds 5, 8 and 9, which respectively incorporate phenethyl moiety, two highly branched aliphatic chains and a short lowly branched chain, were medium potency inhibitors, whereas the remaining ones acted in the micromolar range or did not significantly inhibit the enzyme up to 10000 nM.

Finally the hCA VII inhibition data (depicted in Table 6) show that inhibitory properties of the investigated carbamates against this CA isoform is deeply related to the degree of branching of the aliphatic chains present in the carbamate functionality. Indeed, whereas the bulky, ramificated chain compounds 2 and 3 did not significantly inhibit the enzyme up to 10000 nM, the remaining ones acted as nanomolar inhibitors of hCA VII with K_{I}s spanning in a wide nanomolar range (24.5-865 nM) depending on their substitution pattern.

sec-Butyl (4-sulfamoylphenyl)carbamte (11) is a carbamate derivative of 4-aminobenzenesulfonamide, possessing 4-9 carbons in the aliphatic side chain of the carbamate moieties. sec-Butyl (4-sulfamoylphenyl)carbamate showed a promising anticonvulsant activity in the MES test after ip administration to mice as well as after oral administration to rats with ED₅₀ values of 75 mg/kg and 25 mg/kg, respectively.

1. A compound comprising a 4-sulfamoylphenyl carbamate moiety and an alkyl moiety comprising between 5 and 10 carbon atoms.

2. The compound according to claim 1, wherein the alkyl moiety is a straight or branched alkyl group comprising 5, 6, 7, 8, 9, or 10 carbon atoms.

3. The compound according to claim 1, wherein the alkyl moiety is selected from the group consisting of a substituted or unsubstituted pentyl moiety, a substituted or unsubstituted hexyl moiety, a substituted or unsubstituted heptyl moiety, a substituted or unsubstituted octyl moiety, a substituted or unsubstituted nonyl moiety and a substituted or unsubstituted decyl moiety, provided that the number of carbon atoms in the moiety does not exceed 10 atoms.

4. The compound according to claim 1, wherein the alkyl moiety is selected from the group consisting of 3-methyl-2-propylpentyl, 2-ethylhexyl, 2-propylpentyl, 3,3-dimethylbutyl, phenethyl, 2-ethylbutyl, 2,4,4-trimethylpentyl, 2,4-dimethylpentan-3-yl, 3-methylpentan-2-yl and 3-methylpentyl.

5. The compound according to claim 1, being of formula (I): 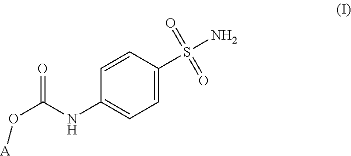
wherein A is an alkyl moiety.

6. The compound according to claim 1, being selected from the group consisting of 3-methyl-2-propylpentyl (4-sulfamoylphenyl)carbamate (1); 2-ethylhexyl (4-sulfamoylphenyl)carbamate (2); 2-propylpentyl (4-sulfamoylphenyl)carbamate (3); 3,3-dimethylbutyl (4-sulfamoylphenyl)carbamate (4); phenethyl (4-sulfamoylphenyl)carbamate (5); 2-ethylbutyl (4-sulfamoylphenyl)carbamate (6); 2,4,4-trimethylpentyl (4-sulfamoylphenyl)carbamate (7); 2,4-dimethylpentan-3-yl (4-sulfamoylphenyl)carbamate (8); 3-methylpentan-2-yl (4-sulfamoylphenyl)carbamate (9) and 3-methylpentyl(4-sulfamoylphenyl)carbamate (10).

7. The compound according to claim 6, being compound (1), (2), (3), (4), (5), (6), (7), (8), (9) or (10).

8. The compound according to claim 6, being compound (1) or (9) or (10).

9. A composition comprising a compound of claim 1.

10. The composition according to claim 9, being a pharmaceutical composition.

11. A method of treatment of a neurological disease or disorder, the method comprising administering to a subject in need of such treatment an effective amount of a compound selected from the group consisting of 3-methyl-2-propylpentyl (4-sulfamoylphenyl)carbamate (1); 2-ethylhexyl (4-sulfamoylphenyl)carbamate (2); 2-propylpentyl (4-sulfamoylphenyl)carbamate (3); 3,3-dimethylbutyl (4-sulfamoylphenyl)carbamate (4); phenethyl (4-sulfamoylphenyl)carbamate (5); 2-ethylbutyl (4-sulfamoylphenyl)carbamate (6); 2,4,4-trimethylpentyl (4-sulfamoylphenyl)carbamate (7); 2,4-dimethylpentan-3-yl (4-sulfamoylphenyl)carbamate (8); 3-methylpentan-2-yl (4-sulfamoylphenyl)carbamate (9), 3-methylpentyl(4-sulfamoylphenyl)carbamate (10) and sec-butyl(4-sulfamoylphenyl)carbamate (11).

12. The method according to claim 11, wherein the neurological disease or disorder is selected from the group consisting of epilepsy, convulsions, seizure disorder, complex partial seizures, status epilepticus, a chemically-induced convulsion and/or seizure disorder, a febrile convulsion condition, pain and psychiatric disorders.

13. An anticonvulsant agent comprising a compound selected from the group consisting of 3-methyl-2-propylpentyl (4-sulfamoylphenyl)carbamate (1); 2-ethylhexyl (4-sulfamoylphenyl)carbamate (2); 2-propylpentyl (4-sulfamoylphenyl)carbamate (3); 3,3-dimethylbutyl (4-sulfamoylphenyl)carbamate (4); phenethyl (4-sulfamoylphenyl)carbamate (5); 2-ethylbutyl (4-sulfamoylphenyl)carbamate (6); 2,4,4-trimethylpentyl (4-sulfamoylphenyl)carbamate (7); 2,4-dimethylpentan-3-yl (4-sulfamoylphenyl)carbamate (8); 3-methylpentan-2-yl (4-sulfamoylphenyl)carbamate (9), 3-methylpentyl(4-sulfamoylphenyl)carbamate (10) and sec-butyl(4-sulfamoylphenyl)carbamate (11).

14. A method of treatment comprising administering to a subject an anticonvulsant agent according to claim 13.

15. A kit comprising an anticonvulsant agent selected from the group consisting of 3-methyl-2-propylpentyl (4-sulfamoylphenyl)carbamate (1); 2-ethylhexyl (4-sulfamoylphenyl)carbamate (2); 2-propylpentyl (4-sulfamoylphenyl)carbamate (3); 3,3-dimethylbutyl (4-sulfamoylphenyl)carbamate (4); phenethyl (4-sulfamoylphenyl)carbamate (5); 2-ethylbutyl (4-sulfamoylphenyl)carbamate (6); 2,4,4-trimethylpentyl (4-sulfamoylphenyl)carbamate (7); 2,4-dimethylpentan-3-yl (4-sulfamoylphenyl)carbamate (8); 3-methylpentan-2-yl (4-sulfamoylphenyl)carbamate (9), 3-methylpentyl(4-sulfamoylphenyl)carbamate (10) and sec-butyl(4-sulfamoylphenyl)carbamate (11).
